(19) United States
(12) Patent Application Publication
(10) Pub. No.: US 2024/0245594 A1
(43) Pub. Date: Jul. 25, 2024

(54) CLEAN BEAUTY EMULSIFICATION SYSTEM

(71) Applicant: AMOREPACIFIC CORPORATION, Seoul (KR)

(72) Inventors: Geunhyuk JANG, Yongin-si (KR); Bongsoo PI, Yongin-si (KR); Jin NAM, Yongin-si (KR); Jaewon YOU, Yongin-si (KR)

(73) Assignee: AMOREPACIFIC CORPORATION, Seoul (KR)

(21) Appl. No.: 18/413,628

(22) Filed: Jan. 16, 2024

(30) Foreign Application Priority Data
Jan. 17, 2023 (KR) 10-2023-0006945

(51) Int. Cl. A61K 8/44; A61K 8/06; A61Q 19/00

(52) U.S. Cl. CPC A61K 8/44; A61K 8/062; A61Q 19/00; A61K 2800/10

(57) ABSTRACT

The present disclosure relates to an emulsifier composition for preparing an oil-in-water emulsion and an oil-in-water emulsion composition that can form a clean beauty emulsion system. In an aspect, since the oil-in-water emulsion composition as disclosed herein does not comprise an emulsifier derived from a synthetic-based polymer, it can provide a non-sticky feel so that customers can use it with confidence.

### CROSS REFERENCE TO RELATED APPLICATION

The present application claims the priority of Korean Patent Application No. 10-2023-0006945, filed on Jan. 17, 2023, the entire contents of which is incorporated herein for all purposes by this reference.

### BACKGROUND OF THE INVENTION

### Field of the Invention

The present disclosure relates to a clean beauty emulsification system, and an emulsifier composition and an oil-in-water emulsion composition for preparing an oil-in-water emulsion.

### Description of the Related Art

Recently, as self-consciousness to actively express oneself has increased due to vigorous social activities, the desire to take care of one's own skin and keep it healthy using cosmetics has increased significantly. At the same time, consumers are highly interest in the effects of various harmful substances caused by environmental pollution and contaminant on their skin. Accordingly, basic research and development is being actively conducted on various forms and formulations of hypoallergenic cosmetics that cause less skin irritation by limiting the use of an emollient, or a raw material or an amount that may cause the skin irritation.

A surfactant or emulsifier (hereinafter referred to as an emulsifier) that can well mix a aqueous phase and an oil phase is absolutely required to prepare an emulsion. The emulsifier has both of a hydrophilic property, which is easily soluble in water, and a hydrophobic property, and is an essential element in preparation of the cosmetics. However, this emulsifier may be irritating to the skin and cause a problem in terms of a customer's confidence.

Further, the emulsifier has a risk of causing environmental pollution during its preparing process or post-processing.

Therefore, many efforts have been made to minimize use of the emulsifier or to utilize a natural emulsifier that is relatively less irritating. However, in this case, it has been difficult to develop a satisfactory product, because emulsification state of the emulsifier was usually unstable, which results in low stability, severe change over time, and hardening of the contents over a long period of time or at a high temperature.

Therefore, there has been a need for developing a natural-based clean beauty emulsification system that is safe for the skin, has an excellent emulsifying power, and does not cause a harmful effect on the environment.

### SUMMARY OF THE INVENTION

An aspect of the present disclosure is to provide an emulsifier composition that does not comprise a synthetic-based polymer and can form a clean beauty emulsification system.

Another aspect of the present disclosure is to provide an oil-in-water emulsion composition that does not comprise an emulsifier derived from the synthetic-based polymer and has a non-sticky feel.

In an aspect, the present disclosure provides an emulsifier composition for preparing an oil-in-water emulsion, comprising myristoyl methyl beta-alanine; and a basic compound.

In another aspect, the present disclosure provides an oil-in-water emulsion composition comprising myristoyl methyl beta-alanine and a basic compound, as an emulsifier.

In an aspect, the emulsifier composition for preparing an oil-in-water emulsion as disclosed herein can provide a clean beauty emulsification system.

In another aspect, since the oil-in-water emulsion composition as disclosed herein does not comprise an emulsifier derived from a synthetic-based polymer, it can provide a non-sticky feel so that customers can use it with confidence.

### BRIEF DESCRIPTION OF THE DRAWINGS

FIG. 1 shows appearance photographs by each prescription of an emulsifier composition for preparing an oil-in-water emulsion according to an embodiment of the present disclosure.

FIGS. 2A to 2F are the results of observing emulsified particles by each prescription of an oil-in-water emulsion composition according to an embodiment of the present disclosure.

### DETAILED DESCRIPTION OF THE INVENTION

Hereinafter, the present disclosure will be described in more detail through the following embodiments. However, the embodiments are intended to provide only for illustrative purposes to aid understanding of the present disclosure without limiting the category and scope of the present disclosure thereto.

### Emulsifier Composition for Preparing Oil-In-Water Emulsions

An exemplary embodiment of the present disclosure provides an emulsifier composition for preparing an oil-in-water emulsion, comprising myristoyl methyl beta-alanine and a basic compound.

According to an embodiment of the present disclosure, the myristoyl methyl beta-alanine (MMbA), a beta-amino acid derivative represented by the following formula, can produce an oil-in-water emulsion under a basic condition, and can form an emulsification system even without using an emulsifier derived from a synthetic-based polymer. Also, since the myristoyl methyl beta-alanine does not use the emulsifier derived from the synthetic-based polymer, it has an advantage of providing a non-sticky feel so that customers can use it with confidence.

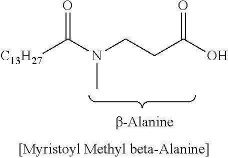

Phytosyeryl/decyltetradecyl myristoyl methyl beta-alanine (P/D MMbA) represented by the following formula, which has a similar structure to the MMbA but has a different functional group, that is, an ester form rather than a carboxylic acid, has been already used for a hair conditioning agent, a skin protecting agent, and moisturizer, as a cosmetic raw material, but has made it difficult to use as an emulsifier due to a poor emulsifying power thereof.

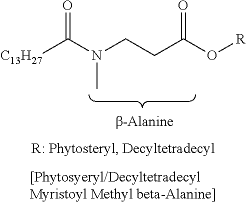

### (Product Name: Eldew APS 307)

Specifically, the P/D MMbA, which has an ester form (—COO—), has no hydrophilic portion and has hydrophobicity increased, so that the P/D MMbA has low solubility in water, making it difficult to use the P/D MMbA as an emulsifier. On the other hand, the MMbA containing a carboxylic acid (—COOH) structure has a hydrophilic property, and especially in case a amino acid is used, the MMbA is modified into a carboxylate (—COO⁻) form to increase the hydrophilic property, making it possible to use the MMbA as the emulsifier.

Further, if the MMbA is used under in a neutral or acidic condition, it may be difficult to use the MMbA as the emulsifier because a gelling occurs or a layer separation occurs due to little emulsification.

In an embodiment, the basic compound may be a basic amino acid. According to an embodiment of the present disclosure, if the basic amino acid is used to create a basic condition, it is possible to prepare a clean beauty emulsification system that does not comprising a synthetic-based polymer.

In an embodiment, the basic compound may be one or more selected from the group consisting of arginine, histidine, and lysine.

In an embodiment, a pH of the emulsifier composition may be 7 to 9.

For example, the pH may be 7 or more, 7.5 or more, 8 or more, or 8.5 or more, and may be 9 or less, 8.5 or less, 8 or less, or 7.5 or less.

However, an oil-in-water emulsion composition comprising the above emulsifier composition makes possible it to adjust the pH to a desired pH without being limited thereto.

In an embodiment, the oil-in-water emulsion may comprise an oil phase and an aqueous phase, wherein the oil phase contains myristoyl methyl beta-alanine, and the aqueous phase contains a basic compound.

In an embodiment, the oil phase may further contain squalane, and the aqueous phase may further contain water.

In an embodiment, a content of the myristoyl methyl beta-alanine may be 0.5 to 20% by weight based on the total weight of the oil-in-water emulsion. In the emulsification system of the present disclosure, the emulsification may be possible even if the MMbA is contained in a small amount.

For example, a content of the myristoyl methyl beta-alanine may be 0.5% by weight or more, 1% by weight or more, 3% by weight or more, 5% by weight or more, 7% by weight or more, 9% by weight or more, 11% by weight or more, 13% by weight or more, 15% by weight or more, 17% by weight or more, or 19% by weight or more, based on the total weight of the oil-in-water emulsion, and may be 20% by weight or less, 18% by weight or less, 16% by weight or less, 14% by weight or less, 12% by weight or less, 10% by weight or less, 8% by weight or less, 6% by weight or less, 3% by weight or less, or 1% by weight or less, based on the total weight of the oil-in-water emulsion.

In an embodiment, a content of the basic compound may be 0.2 to 15% by weight based on the total weight of the oil-in-water emulsion. If the content of the basic compound is less than 0.2% by weight, a carboxylic acid functional group of the MMbA may not be sufficiently modified into a salt thereof, and if it is more than 15% by weight, there may be a problem with solubility or the pH may become higher.

For example, a content of the basic compound may be 0.2% by weight or more, 0.5% by weight or more, 1% by weight or more, 3% by weight or more, 5% by weight or more, 7% by weight or more, 9% by weight or more, 11% by weight or more, or 13% by weight or more, based on the total weight of the oil-in-water emulsion, and may be 15% by weight or less, 13% by weight or less, 11% by weight or less, 9% by weight or less, 7% by weight or less, 5% by weight or less, 3% by weight or less, 1% by weight or less, or 0.5% by weight or less, based on the total weight of the oil-in-water emulsion.

In an embodiment, the oil-in-water emulsion may be a cosmetic composition.

In an embodiment, the cosmetic composition may comprise a cosmetically or dermatologically acceptable medium or substrate thereof. The cosmetic composition may be provided in the form of all formulations suitable for topical application, such as a solution, a gel, a solid, a pasty anhydrous product, an emulsion obtained by dispersing an oil phase into an aqueous phase, a suspension, a microemulsion, a microcapsule, a microgranule, or an ionic (liposome) and non-ionic vesicular dispersion, or in the form of a cream, a skin, a lotion, a powder, an ointment, a spray or a conceal stick. The cosmetic composition may be prepared according to the conventional method well known in the art. The composition according to the present disclosure may also be used in the form of a foam or in the form of an aerosol composition further comprising a compressed propellant.

The cosmetic composition according to the present disclosure is not particularly limited in the formulation thereof, and may be formulated into, for example, a softening lotion, an astringent lotion, a nourishing lotion, a nourishing cream, a massage cream, an essence, an eye cream, an eye essence, a cleansing cream, a cleansing foam, a cleansing water, a cleansing tissue, a pack, a powder, a body lotion, a body cream, a body oil, and a body essence containing the cosmetic composition.

In case the formulation of the cosmetic composition according to the present disclosure is the paste, the cream, or the gel, an animal fiber, a plant fiber, a wax, a paraffin, a starch, a tracant, a cellulose derivative, polyethylene glycol, a silicone, a bentonite, a silica, a talc, zinc oxide, etc. may be used as a carrier component.

In case the formulation of the cosmetic composition according to the present disclosure is the powder or the spray, a lactose, a talc, a silica, aluminum hydroxide, calcium silicate, or a polyamide powder may be used as the carrier component, and especially in the case of the spray, the formulation may further comprise a propellant such as chlorofluorohydrocarbon, propane/butane, or dimethyl ether.

In case the formulation of the cosmetic composition according to the present disclosure is the solution or the emulsion, a solvent, a solvating agent, or an emulsifying agent may be used as the carrier component, and may include water, ethanol, isopropanol, ethyl carbonate, ethyl acetate, benzyl alcohol, benzyl benzoate, propylene glycol, 1,3-butylglycol oil, glycerol fatty ester, polyethylene glycol, or fatty acid ester of sorbitan.

In case the formulation of the cosmetic composition according to the present disclosure is the suspension, the carrier component may include water, a liquid diluent such as ethanol or propylene glycol, a suspending agent such as ethoxylated isostearyl alcohol, polyoxyethylene sorbitol ester and polyoxyethylene sorbitan ester, a microcrystalline cellulose, aluminum metahydroxide, a bentonite, an agar, a tracant, etc.

The cosmetic composition according to the present disclosure may further comprise an functional additive and components added into a general cosmetic composition. The functional additive may include an ingredient selected from the group consisting of a water-soluble vitamin, an oil-soluble vitamin, a high molecular weight peptide, a high molecular weight polysaccharide, a squalane, a sphingo lipid, and a seaweed extract.

In addition to the above-mentioned functional additive, the cosmetic composition according to the present disclosure may also comprise components added into a general cosmetic composition, if necessary. Other components that may be added into the cosmetic composition may include an oil and fat ingredient, a moisturizers, an emollient, an emulsifier, organic and inorganic pigments, an organic powder, a ultraviolet absorber, a preservative, a disinfectant, an antioxidant, a plant extract, a pH adjuster, an alcohol, a pigment, a fragrance, a blood circulation promoter, a cooling agent, a limiting agent, a purified water, etc.

### Oil-In-Water Emulsion Composition

Another exemplary embodiment of the present disclosure provides an oil-in-water emulsion composition comprising myristoyl methyl beta-alanine and a basic compound, as an emulsifier.

According to an embodiment of the present disclosure, the myristoyl methyl beta-alanine (MMbA) of the N-acyl neutral amino acid series and the basic compound may be used as the emulsifier to form the oil-in-water emulsion composition having an outer phase as an aqueous phase and an inner phase as an oil phase. The oil-in-water emulsion composition may be provided without using an emulsifier containing a separate synthetic based-polymer.

In an embodiment, the basic compound may be a basic amino acid.

In an embodiment, the basic compound may be one or more selected from the group consisting of arginine, histidine, and lysine.

In an embodiment, a pH of the emulsifier may be 7 to 9.

For example, the pH may be 7 or more, 7.5 or more, 8 or more, or 8.5 or more, and may be 9 or less, 8.5 or less, 8 or less, or 7.5 or less.

In an embodiment, the oil-in-water emulsion composition may comprise an oil phase; and an aqueous phase, wherein the oil phase contains the myristoyl methyl beta-alanine, and the aqueous phase contains the basic compound.

In an embodiment, the oil phase may further contain squalane, and the aqueous phase may further contain water.

In an embodiment, a content of the myristoyl methyl beta-alanine may be 0.5 to 20% by weight based on the total weight of the composition.

For example, the content of the myristoyl methyl beta-alanine may be 0.5% by weight or more, 1% by weight or more, 3% by weight or more, 5% by weight or more, 7% by weight or more, 9% by weight, 11% by weight or more, 13% by weight or more, 15% by weight or more, 17% by weight or more, or 19% by weight or more, based on the total weight of the oil-in-water emulsion composition, and may be 20% by weight or less, 18% by weight or less, 16% by weight or less, 14% by weight or less, 12% by weight or less, 10% by weight or less, 8% by weight or less, 6% by weight or less, 3% by weight or less, or 1% by weight or less, based on the total weight of the oil-in-water emulsion composition.

In an embodiment, a content of the basic compound may be 0.2 to 15% by weight based on the total weight of the oil-in-water emulsion composition.

For example, the content of the basic compound may be 0.2% by weight or more, 0.5% by weight or more, 1% by weight or more, 3% by weight or more, 5% by weight or more, 7% by weight or more, 9% by weight or more, 11% by weight or more, or 13% by weight or more, based on the total weight of the oil-in-water emulsion composition, and may be 15% by weight or less, 13% by weight or less, 11% by weight or less, 9% by weight or less, 7% by weight or less, 5% by weight or less, 3% by weight or less, 1% by weight or less, or 0.5% by weight or less, based on the total weight of the oil-in-water emulsion composition.

In an embodiment, the composition may be a cosmetic composition. The cosmetic composition is as described above.

Another exemplary embodiment of the present disclosure provides a method for emulsifying an oil-in-water emulsion by adding the above-described emulsifier composition to an oil-in-water emulsion.

Still another exemplary embodiment of the present disclosure provides a non-therapeutic use of the above-described emulsifier composition for emulsifying the oil-in-water emulsion.

Hereinafter, the present disclosure will be described in more detail through the following Examples. However, the following Examples are provided only for illustrative purposes to aid understanding of the present disclosure, and the category and scope of the present disclosure are not limited thereto.

### EXAMPLES

An oil-in-water emulsion composition was prepared as shown in Table 1 below to measure an emulsifying power thereof. A specific preparation method is as follows:
1) dissolving an oil phase and an aqueous phase by heating them to 70° C. or higher, respectively;
2) adding the oil phase to the aqueous phase and forming an emulsion using a homomixer; and
3) degassing and cooling the emulsion.

TABLE 1

| | | Comparative | Comparative | | | Comparative | Comparative |
|---|---|---|---|---|---|---|---|
| (Unit; g) | | Example 1 | Example 2 | Example 2 | Example 1 | Example 3 | Example 4 |
| Oil | Squalane | 37 | 37 | 37 | 37 | 37 | 37 |
| phase | MMbA | 10 | 10 | 10 | 10 | 0 | 0 |
| | P/D | 0 | 0 | 0 | 0 | 10 | 10 |
| | MMbA | | | | | | |
| Aqueous | NaHCO3 | 0 | 0 | 3 | 0 | 1.6 | 0 |
| phase | Arginine | 0 | 0 | 0 | 6.1 | 0 | 3.3 |
| | Citric | 6.1 | 0 | 0 | 0 | 0 | 0 |
| | acid | | | | | | |
| | Water | To 100 | To 100 | To 100 | To 100 | To 100 | To 100 |

| Note | MMbA + | MMbA + | MMbA + | P/DMMbA + |
|---|---|---|---|---|
| | acidic | neutral | basic | basic |

Comparative Example 1: Confirmation of an emulsifying power of the MMbA in an acidic condition

Comparative Example 2: Confirmation of an emulsifying power of the MMbA under a neutral condition

Example 1: Confirmation of an emulsifying power of the MMbA under an inorganic base condition (NaHCO₃)

Example 2: Confirmation of an emulsifying power of the MMbA under an organic base (amino acid) condition (arginine)

Comparative Example 3: Confirmation of an emulsifying power of the P/D MMbA (ester form) under an inorganic base condition (NaHCO₃)

Comparative Example 4: Confirmation of an emulsifying power of the P/D MMbA (ester form) under an organic base (amino acid) condition (arginine)

In present Examples, their effects were confirmed by using arginine, one of the amino acids, in order to promote clean beauty most effectively.

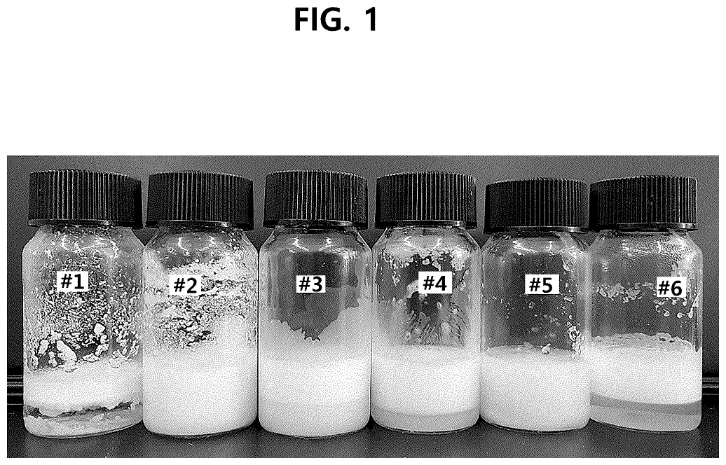

First, from FIG. 1, it is possible to confirm appearance photographs by each prescription in Table 1. The appearance photographs by each prescription can be seen in the order of Comparative Example 1, Comparative Example 2, Example 1, Example 2, Comparative Example 3, and Comparative Example 4, from the left of Table 1.

Referring to them, it was confirmed by Examples 1 and 2 that the myristoyl methyl beta-alanine (MMbA), the beta-amino acid derivative, could produce an oil-in-water emulsion under the basic condition and show that form an emulsification system without using a separate emulsifier.

Further, in the case of the ester form (Comparative Examples 3 and 4), it was confirmed through the appearance photographs that the resultant emulsification system could not be used as an emulsifier, even though it has been already used as a hair conditioning agent, a skin protecting agent, a moisturizer, etc. as a cosmetic raw material.

Furthermore, it was confirmed that even though the MMbA was used, a phenomenon of layer separation occurred under the neutral or acidic condition due to no gelling or little emulsification (Comparative Examples 1 and 2), and that the used MMbA played a role as an excellent emulsifier only under the basic condition.

In particular, it was confirmed that when the arginine, the basic amino acid, was used under the basic condition (Example 2), an oil-in-water composition could be prepared through the clean beauty emulsification system.

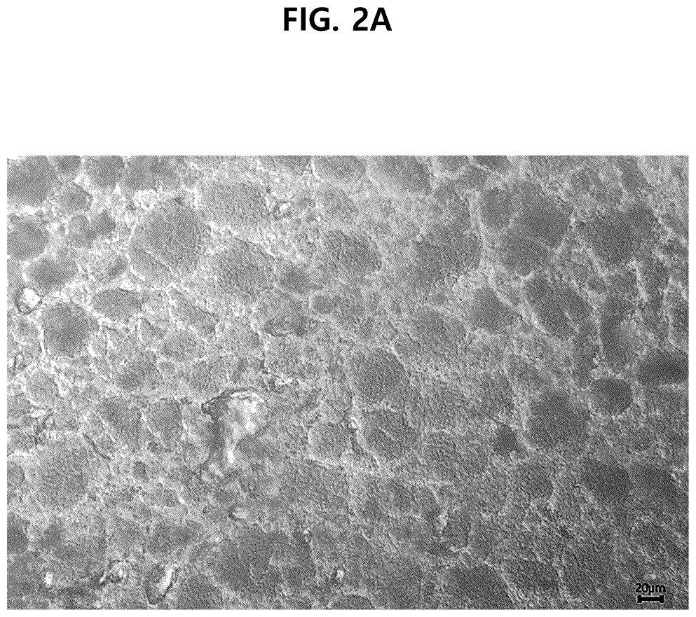

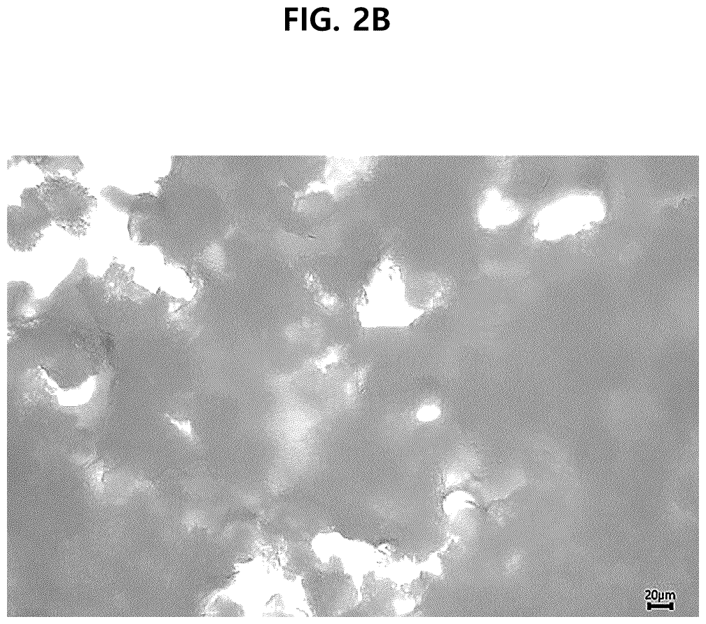

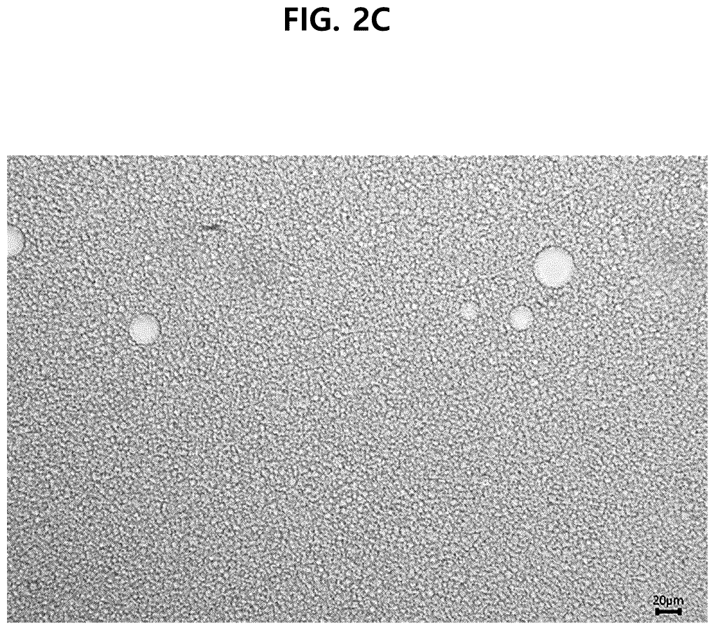

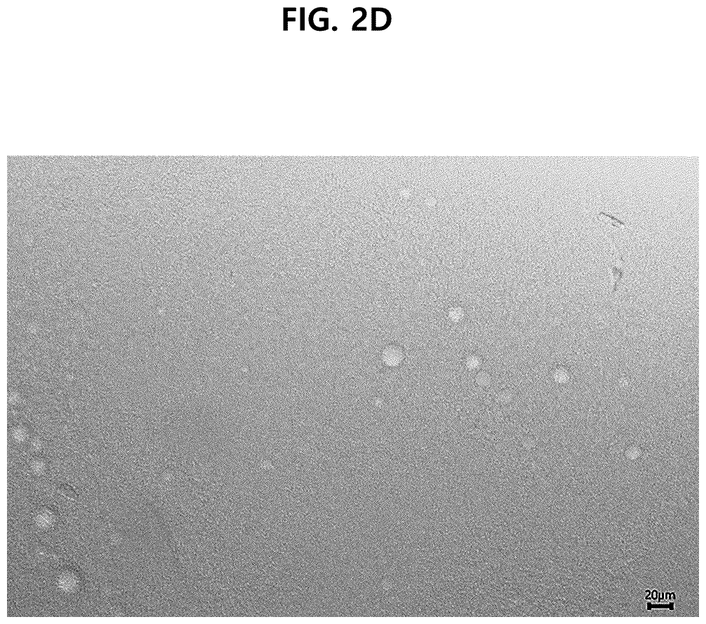

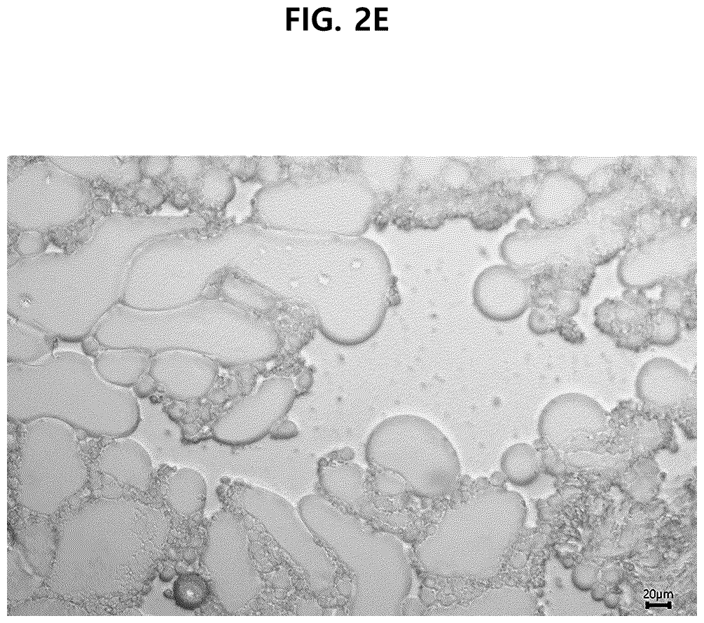

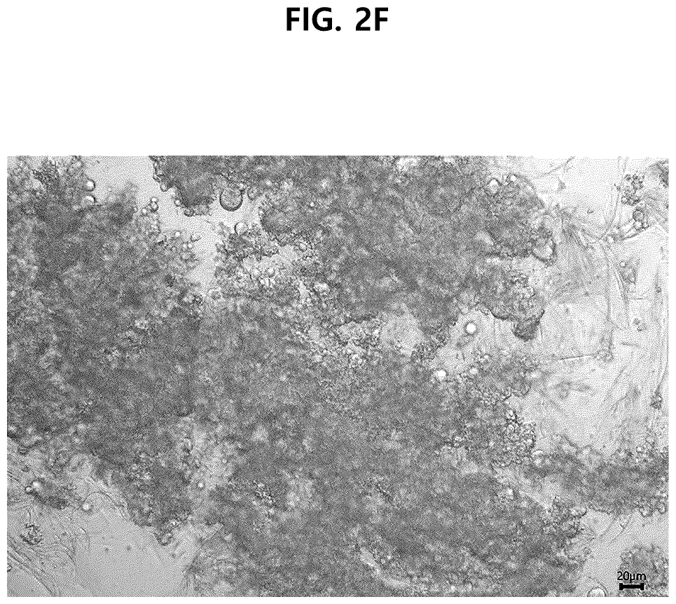

Meanwhile, the results of observing the emulsified particles by each prescription in Table 1 through a microscopic can be confirmed from Table 2 and FIGS. 2A to 2F below.

TABLE 2

| | Comparative | Comparative | | | Comparative | Comparative |
|---|---|---|---|---|---|---|
| | Example 1 | Example 2 | Example 1 | Example 2 | Example 3 | Example 4 |
| Appearance | layer | layer | Creaming | Creaming | layer | layer |
| | separation/ | separation/ | | | separation/ | separation/ |
| | gelling | gelling | | | gelling | gelling |
| Emulsion | X | ⋆ | ★★★★⋆ | ★★★★★ | ★ | ★⋆ |
| formation | | | | | | |
| FIG. | 2A | 2B | 2C | 2D | 2E | 2F |

In Table 2, whether or not the emulsion is formed was evaluated from data obtained by an appearance of the formulation and an observation of the emulsified particles through an optical microscope, which means 0.5 points for a transparent star and 1.0 points for a black star. The above evaluation was made by visually inspecting the microscope images of FIGS. 2A to 2F to determine the relative number and size/distribution of the emulsified particles per a unit area, and a degree of stable formation.

Referring to the above, it could be seen from Examples 1 and 2 that an oil-in-water emulsion with emulsified small particles was formed. Some creaming occurred, but this was caused by the absence of a thickening system that stabilizes the emulsion using only the simple three-component system. This phenomenon has a high possibility of forming a stable emulsion formulation when applying various polymer systems.

Further, it could be confirmed that in the case of Comparative Example 1 (MMbA+acidic condition), almost no emulsification occurred, so the emulsified particles were divided into a transparent aqueous layer and an oil layer, and that in the case of Comparative Example 2 (MMbA+neutral condition), some of the emulsified particles were visible, but the layers were separated due to occurrence of gelling.

Furthermore, in the case of Comparative Examples 3 and 4 (P/DMMbA+base condition), it could be confirmed that a certain level of the emulsified particles was formed, but oil and water were separated due to occurrence of gelling.

What is claimed is:

1. A method for preparing an oil-in-water emulsion, comprising:
a step of adding myristoyl methyl beta-alanine and a basic compound, as an emulsifier, to the oil-in-water emulsion.

2. The method according to claim 1,
wherein the basic compound is a basic amino acid.

3. The method according to claim 1,
wherein the basic compound is one or more selected from the group consisting of arginine, histidine, and lysine.

4. The method according to claim 3,
wherein a pH of the emulsifier is 7 to 9.

5. The method according to claim 1,
wherein the oil-in-water emulsion comprises an oil phase; and an aqueous phase;
wherein the oil phase comprises the myristoyl methyl beta-alanine, and the aqueous phase comprises the basic compound.

6. The method according to claim 1,
wherein a content of the myristoyl methyl beta-alanine is 0.5 to 20% by weight based on the total weight of the oil-in-water emulsion.

7. The method according to claim 1,
wherein a content of the basic compound is 0.2 to 15% by weight based on the total weight of the oil-in-water emulsion.

8. The method according to claim 1,
wherein the oil-in-water emulsion is a cosmetic composition.

9. An oil-in-water emulsion composition comprising myristoyl methyl beta-alanine and a basic compound, as an emulsifier.

10. The oil-in-water emulsion composition according to claim 9,
wherein the basic compound is a basic amino acid.

11. The oil-in-water emulsion composition according to claim 9,
wherein the basic compound is one or more selected from the group consisting of arginine, histidine, and lysine.

12. The oil-in-water emulsion composition according to claim 9,
wherein a pH of the emulsifier is 7 to 9.

13. The oil-in-water emulsion composition according to claim 9,
wherein the oil-in-water emulsion comprises an oil phase; and an aqueous phase;
wherein the oil phase comprises the myristoyl methyl beta-alanine, and the aqueous phase comprises the basic compound.

14. The oil-in-water emulsion composition according to claim 9,
wherein a content of the myristoyl methyl beta-alanine is 5 to 20% by weight based on the total weight of the composition.

15. The oil-in-water emulsion composition according to claim 9,
wherein a content of the basic compound is 1 to 15% by weight based on the total weight of the composition.

16. The oil-in-water emulsion composition according to claim 9,
wherein the composition is a cosmetic composition.
